(19) United States
(12) Patent Application Publication
(10) Pub. No.: US 2005/0249792 A1
(43) Pub. Date: Nov. 10, 2005

(54) Active ingredient release system comprising a plaster containing an active ingredient and at least one active ingredient release regulator

(75) Inventor: Heinrich Kugelmann, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/113,984

(22) Filed: Apr. 26, 2005

(30) Foreign Application Priority Data
Apr. 26, 2004 (DE) 10 2004 020.463.2

(51) Int. Cl. A61K 9/70

(52) U.S. Cl. 424/449

(57) ABSTRACT

An active ingredient release system for the systemic or topical release of an active ingredient into a human or animal organism, including: a) a plaster containing active ingredient, and b) at least one divisible active ingredient release regulator that is impermeable to the active ingredient, or at least one optionally divisible active ingredient release regulator that retards the release of active ingredient and is separate from the plaster, or an optionally divisible active ingredient release regulator that retards the release of active ingredient and is already detachably bonded to the plaster.

### CROSS REFERENCE TO RELATED APPLICATION

This application claims priority from Federal Republic of Germany patent application no. DE 10 2004 020.463.2, filed Apr. 26, 2004, the entire disclosure of which is incorporated herein by reference.

### BACKGROUND OF THE INVENTION

The present invention relates to an active ingredient release system for the systemic or topical release of an active ingredient through and/or onto the skin of a human or animal organism, comprising a plaster containing active ingredient and at least one divisible active ingredient release regulator that is impermeable to the active ingredient, or at least one optionally divisible active ingredient release regulator that retards the release of the active ingredient and is present separate from the plaster, or an optionally divisible active ingredient release regulator that retards the release of active ingredient and that is already detachably bonded to the plaster.

Topical or systemic release of an active ingredient via the skin to a human or animal body normally takes place through a process in which the active ingredient is absorbed by the skin via the contact area with the skin.

The active ingredient is normally released in this way via the entire contact area of the region of a plaster that contains the active ingredient.

To deliver an individual dose of the active ingredient with this type of transdermal administration, in the known matrix plasters containing active ingredient the plaster is normally divided, e.g. by being cut up. This generally leads to dosage inaccuracies. The delivery of an individual dose from plasters that contain the active ingredient in a reservoir is not possible in this way.

This also applies to the individual dosage capability of plasters that contain the active ingredient embedded in a matrix, as described in U.S. 2003/0125659. A disadvantage of the dosing systems described in this published patent application is that the dosage is only appropriate for plasters of a particular predetermined width that in fact corresponds to the dosing device. The various commercially available, arbitrarily shaped matrix plasters containing active ingredient cannot be used according to this known dosing system.

Furthermore, DE 197 33 981 discloses a transdermal therapeutic system for delivering an individual dose, in which a part of the region containing active ingredient of a matrix plaster is covered with a layer that is impermeable to the active ingredient. The system makes provision for a one-off reduction of the individual dose of the transdermal active ingredient release.

### SUMMARY OF THE INVENTION

It was therefore the object of the invention to provide an improved active ingredient release system for controlling release of an active agent from a plaster.

Another object was to provide an active ingredient release system for arbitrarily shaped matrix or reservoir plasters containing active ingredient, which makes it possible to deliver an individual dose of the active ingredient that is appropriate to the patient, e.g. that corresponds to his body weight.

A further object is to provide an active ingredient release system for controlling release of an active agent from a plaster which is simple to handle.

An additional object of the invention was to provide an active ingredient release system in which it is possible to reduce the dosage to a variable extent and/or to administer the active ingredient with an individual release of the active ingredient appropriate to the patient's needs using a single active ingredient release regulator.

These and other objects have been achieved in accordance with the present invention by providing a n active ingredient release system for systemic or topical release of an active ingredient to or through the skin of a human or animal organism, said release system comprising:
a) a plaster containing the active ingredient, and
b) an active ingredient release regulator selected from the group consisting of:
at least one divisible active ingredient release regulator that is impermeable to the active ingredient;
at least one optionally divisible active ingredient release regulator that retards the release of the active ingredient and is present separate from the plaster; and
an optionally divisible active ingredient release regulator that retards the release of active ingredient and that is detachably connected to the plaster.

Thus, in accordance with the present invention, an active ingredient release system for the systemic or topical release of an active ingredient through and/or onto the skin of a human or animal organism, is provided which comprises:
a) a plaster containing active ingredient, and
b) at least one divisible active ingredient release regulator that is impermeable to the active ingredient or at least one optionally divisible active ingredient release regulator that retards the release of active ingredient and is separate from the plaster, or an optionally divisible active ingredient release regulator that retards the release of active ingredient and is already detachably bonded to the plaster.

Preferably, the divisible active ingredient release regulator present is separate from the plaster and is impermeable to the active ingredient and has the following layer structure:
a detachable protective layer,
an adhesive layer,
an active ingredient barrier layer,
optionally a second adhesive layer, and
optionally a second detachable protective layer.

The optionally present second adhesive layer serves to bond the assembled plaster and release regulating system to the skin of a user during application.

In this connection, after removal of the detachable protective layer the desired, individually adapted area of the region of the plaster that contains the active ingredient is covered with the active ingredient release regulator according to the invention containing a barrier layer, and is adhered thereto by the adhesive layer.

Advantageously, this active ingredient release regulator that is impermeable to the active ingredient also has a second adhesive layer on the opposite side of the barrier layer for bonding the assembly to the skin of a user after removal of a second detachable protective layer, so that the active ingredient barrier layer is present between two adhesive layers, and an interruption of contact between the plaster and the user's skin is thereby prevented.

An active ingredient release regulator of this type will preferably also include a second or further detachable protective layer bonded to the second or further adhesive layer and in order to facilitate handling when removing a part of the active ingredient release regulator for delivering an individualized dose of the active ingredient, i.e. for adjusting the variably reduced dosage of the active ingredient. This handling is additionally facilitated by the fact that the active ingredient release regulator is packaged separately from the plaster.

As already mentioned, the release of the active ingredient can be blocked to any desired extent using the separately packaged, divisible active ingredient release regulator which is impermeable to active ingredient, by individual division of the regulator, preferably by appropriately covering all or a desired part of the region of the plaster containing active ingredient. The rate of administration of the active ingredient can thereby be individually metered.

Alternatively, instead of an active ingredient release regulator impermeable to active ingredient, the active ingredient release system according to the invention may comprise an optionally divisible active ingredient release regulator that retards the release of active ingredient. An optionally divisible active ingredient release regulator of this type which retards the release of the active ingredient preferably comprises several layers and has the following structure:
a detachable protective layer,
an adhesive layer that optionally retards the release of active ingredient,
optionally a barrier layer that optionally further retards the release of active ingredient
optionally a second adhesive layer, and
optionally a second detachable protective layer.

Preferably, the optionally divisible active ingredient release regulator according to the invention, which retards the release of the active ingredient, comprises several layers and has the following structure:
a detachable protective layer,
an adhesive layer,
a barrier layer that retards the release of the active ingredient
a second adhesive layer, and
a second detachable protective layer.

If it is separate from the plaster, the optionally divisible active ingredient release regulator according to the invention that retards the release of the active ingredient, is provided with a detachable protective layer disposed over the top of the adhesive layer. This facilitates the handling of the regulator, especially when the layer is being divided. The second adhesive layer is provided in order to bond the plaster to the skin of a human or animal organism after removal of the overlying second protective layer during application.

Accordingly, if it is separate from the plaster, the optionally divisible active ingredient release regulator according to the invention that retards the release of the active ingredient, comprises the following layer structure:
a detachable protective layer,
an adhesive layer,
a barrier layer that retards the release of the active ingredient,
a second adhesive layer, and
a second detachable protective layer.

If the two adhesive layers and are sufficiently thick, they can act as a corresponding layer that retards the release of active ingredient, and the separate barrier layer which retards the release of active ingredient can be omitted.

This naturally applies also to the structure of an optionally divisible active ingredient release regulator that retards the release of the active ingredient in the case where the regulator is already detachably bonded to a plaster. An active ingredient release regulator of this type preferably comprises the following layer structure:
a detachable protective layer that is detachably bonded at least to a partial region of the plaster, preferably to at least the region of the plaster that contains active ingredient,
an adhesive layer detachably connected to the opposite surface of the detachable protective layer,
a barrier layer adjoining the adhesive layer and which retards the release of active ingredient,
a second adhesive layer on the opposite side of the barrier layer, and
a second detachable protective layer disposed over the second adhesive layer.

Preferably, for better handling, the optionally divisible active ingredient regulator already detachably bonded to the plaster comprises the optional second detachable protective layer, as the provision of this second protective layer particularly facilitates handling of the regulator during division thereof.

To apply the active ingredient release system according to the invention in the case where the regulator is separate from the plaster, the detachable protective layer is first removed from the regulator, optionally after the regulator has been divided, and the now exposed adhesive layer is bonded at least to a part of the region of the plaster that contains active ingredient.

If, on the other hand, a regulator that is already detachably bonded to the plaster is used, the regulator is first removed in total from the plaster, optionally divided if desired, the protective layer is removed, and the regulator is reattached by bonding the then exposed adhesive layer to at least a part of the region of the plaster containing active ingredient.

In either event, before application of the plaster to a patient, the second detachable protective layer is then removed from the second adhesive layer so that also this retarded region of the plaster can be brought into direct contact with the skin.

Both the plaster containing the active ingredient, and the active ingredient release regulator, can have any desired shape, size and color. For example, independently of one another, they can have a round, oval, rectangular or other, irregular shape. Preferably, the plaster and the active ingredient release regulator of the active ingredient release system according to the invention have matching shapes. The size of an active ingredient release regulator according to the invention can preferably represent up to 100% of the area of the region of the plaster that contains the active ingredient.

If the active ingredient release regulator present separate from the plaster includes an active ingredient barrier layer, the size of the regulator can correspond to the entire region of the plaster that contains the active ingredient, since the regulator is divided up to yield the desired size corresponding to the desired individual dosage. Preferably, 90% to 10% of the region of the plaster that contains the active ingredient is then covered by the active ingredient release regulator, thereby making it possible to reduce the release of active ingredient by 10 to 90%. The divisibility of the active ingredient release regulator provided with an active ingredient barrier layer means that the individually desirable dose can be adjusted in a simple manner, this being facilitated still further by an active ingredient release regulator separate from the plaster.

The size of an active ingredient release regulator that retards the release of the active ingredient can represent up to 100% of the entire region of a plaster that contains the active ingredient. In one preferred embodiment the possible coverage is 100%. In a further preferred embodiment such an active ingredient release regulator is divisible in order optionally to achieve an active ingredient release that is not retarded by the active ingredient regulator, at least in parts of the region of the plaster that contains active ingredient. To improve handling the agent retarding the release of active ingredient is separate from the plaster, and is preferably packaged separately.

If the active ingredient release regulator is provided with a retarding layer, it is possible to have not only a divisible active ingredient release regulator, but also several such active ingredient release regulators, separate from one another, that are of different sizes. These different sizes then correspond in each case to the desired part to be covered, and hence retarded, of the region of the plaster that contains active ingredient. The different sizes of the active ingredient release regulators packaged separately from one another and from the plaster are then preferably dimensioned in such a way that 10% to 90% of the region of the plaster that contains active ingredient is covered, so that a retarded release of the active ingredient is thereby achieved.

To facilitate the separation of the partial regions of a divisible active ingredient release regulator, the latter may preferably be provided with specified separating means. These separating means preferably take the form of lines of weakness, perforations or cutting marks.

Preferably, the parts of a divisible active ingredient release regulator that can be separated from one another, or the active ingredient release regulators that are separate from one another, carry different distinguishing marks. These distinguishing marks may be comprised of a different coloring and/or coding of the parts that can be separated from one another or the regulators packed separately from one another.

Preferably, the active ingredient release system according to the invention is made available as a kit comprising the plaster containing active ingredient, and one or more active ingredient release regulators. If the active ingredient release regulator is not already detachably bonded to the plaster, it is preferably packaged separately from the plaster as a component of the kit according to the invention.

The active ingredient release system according to the invention is suitable for administering any systemically or topically administrable active ingredient. Preferably the active ingredient release system according to the invention is suitable for the transdermal and/or topical administration of all types of pharmaceutical active ingredients. In particular the plaster is suitable for the transdermal release of at least one pharmaceutical active ingredient selected from the group consisting of analgesics, local anesthetics, hormones, contraceptives, inocula, immune modulators, antiallergics, antihistamines, cardiac agents, antihypertensive agents, psychopharmaceuticals, anti-rheumatics and enzymes. The release system of the invention is particularly well suited for administration of at least one pharmaceutical active ingredient selected from the group consisting of narcotics; tranquillizers, preferably benzodiazepines; stimulants; and other anaesthetics.

Particularly preferably the release system according to the invention is suitable for the administration of at least one opioid, tranquilizer or other anesthetic capable of transdermal administration and selected from the group consisting of:
N-{1-[2-(4-ethyl-5-oxo-2-tetrazolin-1-yl)ethyl]-4-methoxymethyl-4-piperidyl}propionanilide (alfentanil),
5,5-diallylbarbituric acid (allobarbital),
allylprodine,
alphaprodine,
8-chloro-1-methyl-6-phenyl-4H-[1,2,4]-triazolo[4,3-a][1,4]-benzodiazepine (alprazolam),
2-diethylaminopropiophenone (amfepramone),
(±)-α-methylphenethylamine (amphetamine),
2-α-methylphenethylamino)-2-phenylacetonitrile (amphetaminil),
5-ethyl-5-isopentylbarbituric acid (amobarbital),
anileridine,
apocodeine,
5,5-diethylbarbituric acid (barbital),
benzylmorphine,
bezitramide,
7-bromo-5-(2-pyridyl)-1H-1,4-benzodiazepin-2 (3H)-one (bromazepam),
2-bromo-4-(2-chlorophenyl)-9-methyl-6H-thieno[3,2-f][1,2,4]triazolo[4,3-a][1,4]diazepine (brotizolam),
17-cyclopropylmethyl-4,5α-epoxy-7α[(S)-1-hydroxy-1,2,2-trimethylpropyl]-6-methoxy-6,14-endo-ethanomorphinan-3-ol (buprenorphine),
5-butyl-5-ethylbarbituric acid (butobarbital),
butorphanol,
(7-chloro-1,3-dihydro-1-methyl-2-oxo-5-phenyl-2H-1,4-benzodiazepin-3-yl) dimethylcarbamate (camazepam),
(1S,2S)-2-amino-1-phenyl-1-propanol (cathine/D-norpseudoephedrine),
7-chloro-N-methyl-5-phenyl-3H-1,4-benzodiazepin-2-ylamine 4-oxide (chlordiazepoxide),
7-chloro-1-methyl-5-phenyl-1H-1,5-benzodiazepine-2,4(3H, 5H)-dione (clobazam),
5-(2-chlorophenyl)-7-nitro-1H-1,4-benzodiazepin-2 (3H)-one (clonazepam),
clonitazene,
7-chloro-2,3-dihydro-2-oxo-5-phenyl-1H-1,4-benzodiazepine-3-carboxylic acid (clorazepate),
5-(2-chlorophenyl)-7-ethyl-1-methyl-1H-thieno[2,3-e][1,4]diazepin-2 (3H)-one (clotiazepam),
10-chloro-11b-(2-chlorophenyl)-2,3,7,11b-tetrahydrooxazolo[3,2-d][1,4]benzodiazepin-6 (5H)-one (cloxazolam),
(−)-methyl[3β-benzoyloxy-2β(1αH,5αH)-tropanecarboxylate] (cocaine),
4,5α-epoxy-3-methoxy-17-methyl-7-morphinen-6α-ol (codeine),
5-(1-cyclohexenyl)-5-ethylbarbituric acid (cyclobarbital),
cyclorphan,
cyprenorphine,
7-chloro-5-(2-chlorophenyl)-1H-1,4-benzodiazepin-2 (3H)-one (delorazepam),
desomorphine,
dextromoramide,
(+)-(1-benzyl-3-dimethylamino-2-methyl-1-phenylpropyl)propionate (dextropropoxyphene),
dextromethorphan,
dezocine,
diampromide,
diamorphone,
7-chloro-1-methyl-5-phenyl-1H-1,4-benzodiazepin-2 (3H)-one (diazepam),
4,5α-epoxy-3-methoxy-17-methyl-6α-morphinanol (dihydrocodeine)
4,5α-epoxy-17-methyl-3,6α-morphinandiol (dihydromorphine),
dimenoxadol,
dimepheptanol,
dimethylthiambutene,
dioxaphetyl butyrate,
dipipanone,
(6aR,10aR)-6,6,9-trimethyl-3-pentyl-6a,7,8,10a-tetrahydro-6H-benzo [c]-chromen-1-ol (dronabinol),
eptazocine,
8-chloro-6-phenyl-4H-[1,2,4]triazolo[4,3-a][1,4]benzodiazepine (estazolam),
ethoheptazine,
ethylmethylthiambutene,
ethyl[7-chloro-5-(2-fluorophenyl)-2,3-dihydro-2-oxo-1H-1,4-benzodiazepine-3-carboxylate](ethyl loflazepate),
4,5α-epoxy-3-ethoxy-17-methyl-7-morphinen-6α-ol (ethylmorphine), etonitazene,
4,5α-epoxy-7α-(1-hydroxy-1-methylbutyl)-6-methoxy-17-methyl-6,14-endo-ethenomorphinan-3-ol (etorphine),
N-ethyl-3-phenyl-8,9,10-trinorbornan-2-ylamine (fencamfamine),
7-[2-α-methylphenethylamino)ethyl]theophylline (fenethylline),
3-(α-methylphenethylamino)propionitrile (fenproporex),
N-(1-phenethyl-4-piperidyl)propionanilide (fentanyl),
7-chloro-5-(2-fluorophenyl)-1-methyl-1H-1,4-benzodiazepin-2 (3H)-one (fludiazepam),
5-(2-fluorophenyl)-1-methyl-7-nitro-1H-1,4-benzodiazepin-2 (3H)-one (flunitrazepam),
7-chloro-1-(2-diethylaminoethyl)-5-(2-fluorophenyl)-1H-1,4-benzodiazepin-2(3H)-one (flurazepam),
7-chloro-5-phenyl-1-(2,2,2-trifluoroethyl)-1H-1,4-benzodiazepin-2 (3H)-one (halazepam),
10-bromo-11b-(2-fluorophenyl)-2,3,7,11b-tetrahydro[1,3]oxazolo[3,2-d][1,4]benzodiazepin-6 (5H)-one (haloxazolam),
heroin,
4,5α-epoxy-3-methoxy-17-methyl-6-morphinanone (hydrocodone),
4,5α-epoxy-3-hydroxy-17-methyl-6-morphinanone (hydromorphone),
hydroxypethidine,
isomethadone,
hydroxymethylmorphinan,
11-chloro-8,12b-dihydro-2,8-dimethyl-12b-phenyl-4H-[1,3]oxazino[3,2-d][1,4]benzodiazepine-4,7 (6H)-dione (ketazolam),
1-[4-(3-hydroxyphenyl)-1-methyl-4-piperidyl]-1-propanone (ketobemidone),
(3S,6S)-6-dimethylamino-4,4-diphenylheptan-3-yl acetate (levoacetylmethadol (LAAM)),
(−)-6-dimethylamino-4,4-diphenyl-3-heptanone (levomethadone),
(−)-17-methyl-3-morphinanol (levorphanol),
levophenacylmorphan,
levoxemacine,
lofentanil,
6-(2-chlorophenyl)-2-(4-methyl-1-piperazinylmethylene)-8-nitro-2H-imidazo[1,2-a][1,4]benzodiazepin-1(4H)-one (loprazolam),
7-chloro-5-(2-chlorophenyl)-3-hydroxy-1H-1,4-benzodiazepin-2 (3H)-one (lorazepam),
7-chloro-5-(2-chlorophenyl)-3-hydroxy-1-methyl-1H-1,4-benzodiazepin-2 (3H)-one (lormetazepam),
5-(4-chlorophenyl)-2,5-dihydro-3H-imidazo[2,1-α]isoindol-5-ol (mazindol),
7-chloro-2,3-dihydro-1-methyl-5-phenyl-1H-1,4-benzodiazepine (medazepam),
N-(3-chloropropyl)-α-methylphenethylamine (mefenorex),
meperidine,
2-methyl-2-propyltrimethylene dicarbamate (meprobamate),
meptazinol,
metazocine,
methylmorphine,
N,α-dimethylphenethylamine (metamphetamine),
(±)-6-dimethylamino-4,4-diphenyl-3-heptanone (methadone),
2-methyl-3-o-tolyl-4 (3H)-quinazolinone (methaqualone),
methyl[2-phenyl-2-(2-piperidyl)acetate](methylphenidate),
5-ethyl-1-methyl-5-phenylbarbituric acid (methylphenobarbital),
3,3-diethyl-5-methyl-2,4-piperidinedione (methyprylon),
metopon, 8-chloro-6-(2-fluorophenyl)-1-methyl-4H-imidazo[1,5-α][1,4]benzodiazepine (midazolam),
2-(benzhydrylsulfinyl)acetamide (modafinil),
4,5α-epoxy-17-methyl-7-morphinene-3,6α-diol (morphine),
myrophine,
(±)-trans-3-(1,1-dimethylheptyl)-7,8,10,10α-tetrahydro-1-hydroxy-6,6-dimethyl-6H-dibenzo[b,d]pyran-9(6αH)-one (nabilone),
nalbuphen,
nalorphine,
narceine,
nicomorphine,
1-methyl-7-nitro-5-phenyl-1H-1,4-benzodiazepin-2 (3H)-one (nimetazepam),
7-nitro-5-phenyl-1H-1,4-benzodiazepin-2 (3H)-one (nitrazepam),
7-chloro-5-phenyl-1H-1,4-benzodiazepin-2 (3H)-one (nordazepam),
norlevorphanol,
6-dimethylamino-4,4-diphenyl-3-hexanone (normethadone),
normorphine,
norpipanone,
the coagulated sap of plants belonging to the species Papaver somniferum (opium),
7-chloro-3-hydroxy-5-phenyl-1H-1,4-benzodiazepin-2 (3H)-one (oxazepam),
(cis, trans)-10-chloro-2,3,7,11b-tetrahydro-2-methyl-11b-phenyloxazolo[3,2-d][1,4]benzodiazepin-6-(5H)-one (oxazolam),
4,5α-epoxy-14-hydroxy-3-methoxy-17-methyl-6-morphinanone (oxycodone),
oxymorphone,
plants and parts of plants belonging to the species Papaver somniferum (including the subspecies setigerum),
papaveretum,
2-imino-5-phenyl-4-oxazolidinone (pernoline),
1,2,3,4,5,6-hexahydro-6,11-dimethyl-3-(3-methyl-2-butenyl)-2,6-methano-3-benzazocin-8-ol (pentazocine),
5-ethyl-5-(1-methylbutyl)barbituric acid (pentobarbital), ethyl(1-methyl-4-phenyl-4-piperidinecarboxylate) (pethidine),
phenadoxone,
phenomorphan,
phenazocine,
phenoperidine,
piminodine,
pholcodine,
3-methyl-2-phenylmorpholine (phenmetrazine),
5-ethyl-5-phenylbarbituric acid (phenobarbital), α,α-dimethylphenethyl-amine (phentermine),
7-chloro-5-phenyl-1-(2-propynyl)-1H-1,4-benzodiazepin-2 (3H)-one (pinazepam),
α-(2-piperidyl)benzhydryl alcohol (pipradrol),
1′-(3-cyano-3,3-diphenylpropyl) [1,4′-bipiperidine]-4′-carboxamide (piritramide),
7-chloro-1-(cyclopropylmethyl)-5-phenyl-1H-1,4-benzodiazepin-2 (3H)-one (prazepam),
premethadione,
profadol,
proheptazine,
promedol,
properidine,
propoxyphene,
N-(1-methyl-2-piperidinoethyl)-N-(2-pyridyl)propionamide, methyl {3-[4-methoxycarbonyl-4-(N-phenylpropanamido)piperidino]propanoate}(remifentanil),
5-sec-butyl-5-ethylbarbituric acid (secbutabarbital),
5-allyl-5-(1-methylbutyl)barbituric acid (secobarbital),
N-{4-methoxymethyl-1-[2-(2-thienyl)ethyl]-4-piperidyl}propionanilide (sufentanil),
7-chloro-2-hydroxymethyl-5-phenyl-1H-1,4-benzodiazepin-2 (3H)-one (temazepam),
7-chloro-5-(1-cyclohexenyl)-1-methyl-1H-1,4-benzodiazepin-2 (3H)-one (tetrazepam),
ethyl(2-dimethylamino-1-phenyl-3-cyclohexene-1-carboxylate) (tilidine (cis and trans)),
tramadol,
8-chloro-6-(2-chlorophenyl)-1-methyl-4H-[1,2,4]triazolo[4,3-a][1,4]benzodiazepine (triazolam),
5-(1-methylbutyl)-5-vinylbarbituric acid (vinylbital),
(1R*,2R*)-3-(3-dimethylamino-1-ethyl-2-methylpropylphenol,
(1R,2R,4S)-2-(dimethylamino)methyl-4-(p-fluorobenzyloxy)-1-(m-methoxy-phenyl)cyclohexanol,
(1R,2R)-3-(2-dimethylaminomethylcyclohexyl)phenol,
(1S,2S)-3-(3-dimethylamino-1-ethyl-2-methylpropyl)phenol,
(2R,3R)-1-dimethylamino-3-(3-methoxyphenyl)-2-methylpentan-3-ol,
(1RS,3RS,6RS)-6-dimethylaminomethyl-1-(3-methoxyphenyl)cyclohexane-1,3-diol,
3-(2-dimethylaminomethyl-1-hydroxycyclohexyl)phenyl 2-(4-isobutylphenyl)propionate,
3-(2-dimethylaminomethyl-1-hydroxycyclohexyl)phenyl 2-(6-methoxynaphthalen-2-yl)propionate,
3-(2-dimethylaminomethylcyclohex-1-enyl)phenyl 2-(4-isobutylphenyl)-propionate,
3-(2-dimethylaminomethylcyclohex-1-enyl)phenyl 2-(6-methoxynaphthalen-2-yl)propionate,
(RR,SS)-2-acetoxy-4-trifluoromethylbenzoic acid 3-(2-dimethylaminomethyl-1-hydroxycyclohexyl)phenyl ester,
(RR,SS)-2-hydroxy-4-trifluoromethylbenzoic acid 3-(2-dimethylamino-methyl-1-hydroxycyclohexyl)phenyl ester,
(RR,SS)-4-chloro-2-hydroxybenzoic acid 3-(2-dimethylaminomethyl-1-hydroxycyclohexyl)phenyl ester,
(RR,SS)-2-hydroxy-4-methylbenzoic acid 3-(2-dimethylaminomethyl-1-hydroxycyclohexyl)phenyl ester,
(RR,SS)-2-hydroxy-4-methoxybenzoic acid 3-(2-dimethylaminomethyl-1-hydroxycyclohexyl)phenyl ester,
(RR,SS)-2-hydroxy-5-nitrobenzoic acid 3-(2-dimethylaminomethyl-1-hydroxycyclohexyl)phenyl ester, and
(RR,SS)-2′,4′-difluoro-3-hydroxybiphenyl-4-carboxylic acid 3-(2-dimethylaminomethyl-1-hydroxycyclohexyl)phenyl ester,
as well as their corresponding stereoisomeric compounds, their respective derivatives, especially amides, esters or ethers, and their respective physiologically acceptable compounds, especially their salts and solvates, particularly preferably their hydrochlorides.

The compounds (1R*,2R*)-3-(3-dimethylamino-1-ethyl-2-methylpropyl)phenol, (1R,2R,4S)-2-(dimethylamino)methyl-4-(p-fluorobenzyloxy)-1-(m-methoxyphenyl)cyclohexanol or their stereoisomeric compounds or their physiologically acceptable compounds, especially their hydrochlorides, their derivatives such as esters, ethers or amides, and processes for their preparation, are disclosed e.g. in U.S. Pat. No. 6,248,737 (=EP 693,475) or U.S. Pat. No. 5,801,201 (=EP 780,369), the disclosures of which are incorporated herein by reference.

Opioids are preferably used as active ingredients for transdermal administration. Particularly preferred active ingredients may be selected from the group consisting of morphine, oxycodone, buprenorphine and fentanyl, their derivatives, preferably esters, ethers or amides, or their respective physiologically acceptable compounds, preferably their salts or solvates, particularly preferably their hydrochlorides.

Preferably the concentration of the active ingredient is its saturation concentration or slightly less than the saturation concentration, since the release to the skin is thereby promoted. This saturation concentration may be determined by routine tests.

The active ingredient barrier layer of the active ingredient release regulator according to the invention preferably is comprised of a polymer such as a polyester, e.g. polyethylene terephthalate, polyolefin, e.g. polyethylene, polypropylene or polybutylene, polycarbonate, polyethylene oxide, polyurethane, polystyrene, polyamide, polyimide, polyvinyl acetate, polyvinyl chloride or polyvinylidene chloride, and/or a copolymer of e.g. acrylonitrile/butadiene/styrene. The thickness of the layer is preferably 5 to 25 μm.

The layer of an active ingredient release regulator according to the invention that retards the release of the active ingredient can be comprised of a film-forming polymer selected from the group consisting of cellulose derivatives such as ethyl cellulose, hydroxypropyl cellulose or carboxymethyl cellulose, polyethylenes, chlorinated polyethylenes, polypropylenes, polyurethanes, polycarbonates, polyacrylic acid esters, polyacrylates, polymethacrylates, polyvinyl alcohols, polyvinyl chlorides, polyvinylidene chlorides, polyvinylpyrrolidones, polyethylene terephthalates, polytetrafluoroethylenes, ethylene/propylene copolymers, ethylene/ethyl acrylate copolymers, ethylene/vinyl acetate copolymers, ethylene/vinyl alcohol copolymers, vinyl chloride/vinyl acetate copolymers, vinylpyrrolidone/ethylene/vinyl acetate copolymers, rubbers, rubber-like synthetic homopolymers, copolymers or block polymers, silicones, silicone derivatives and mixtures thereof.

As a layer that retards the release of the active ingredient, it is preferable to use a layer based on an ethylene/vinyl acetate copolymer, a polyacrylate, or a layer formed from two adhesive layers with the omission of a separate layer that retards the release of the active ingredient, said layer retarding the release of the active ingredient for the predetermined life of the plaster, preferably 3 to 7 days. Conventional active ingredient reservoir membranes can also be used as a retarding layer.

It is possible to provide several retarding active ingredient release regulators separate from the plaster and from one another, each exhibiting a different degree of retardation, preferably in the same kit, so that the appropriate active ingredient release regulator for the desired release rate can be selected from several active ingredient release regulators with different retardation properties.

If the active ingredient release regulator that acts in a retarding manner is already detachably bonded to the plaster, the detachable protective layer (1) is impermeable to active ingredient, at least in the area in which the region of the plaster containing active ingredient is covered, so that during storage of the transdermal active ingredient release system according to the invention the active ingredient remains in the region of the plaster containing active ingredient. Direct or indirect contact between the region of the plaster containing active ingredient and the retarding agent is then possible only immediately before the application of the plaster and after removal of the detachable protective layer (1).

The plaster containing active ingredient can be built up in accordance with the reservoir or matrix system (Bauer K. H., Frömming K.-H., Führer C., Pharmazeutische Technologie, pages 381-383; Müller R. H., Hildebrand G. E., Pharmazeutische Technologie: Moderne Arzneiformen, Chapter 8). According to the matrix system, the plaster containing active ingredient can preferably comprise a carrier layer, a layer containing active ingredient, and an adhesive layer, it being possible for the layer containing the active ingredient to be at the same time the adhesive layer, in which the active ingredient is dissolved and/or dispersed in a matrix together with the adhesive. If the plaster containing the active ingredient and the active ingredient release regulator are separate before application, the plaster additionally comprises a detachable protective layer.

The adhesives used for the adhesive layers of the plaster and the active ingredient release regulator are preferably pressure-sensitive adhesives. Examples of suitable adhesives include polymers such as polyacrylates, polyvinyl ethers, polyisobutylenes (PIB), styrene/isoprene or butadiene/styrene copolymers or polyisoprene rubbers. Silicone adhesives, e.g. optionally crosslinked polydimethylsiloxanes, are also suitable. Other suitable adhesives include synthetic resins composed of esters of glycines, glycerol or pentaerythritol, or of hydrocarbons such as polyterpenes. Polyacrylate-based adhesives are prepared by polymerizing acrylates, methacrylates, alkyl acrylates and/or alkyl methacrylates, optionally with other α,β-unsaturated monomers such as acrylamide, dimethylacrylamide, dimethylaminoethyl acrylate, hydroxyethyl acrylate, hydroxypropyl acrylate, methoxyethyl acrylate, methoxyethyl methacrylate, acrylonitrile and/or vinyl acetate.

The adhesive layers of the plaster and the active ingredient release regulator can also contain skin penetration enhancers, fillers (such as zinc oxide or silica), crosslinking agents, antioxidants and/or solvents. The thickness of the adhesive layers is preferably 3 to 100 μm in each case.

The carrier layer or covering layer of the plaster is preferably impermeable and inert to the substances present in the layer containing active ingredient and in the adhesive layer, especially to the active ingredient which is to be transdermally and/or topically released, and can be composed of polymers such as polyesters, e.g. polyethylene terephthalate, polyolefins, e.g. polyethylenes, polypropylenes or polybutylenes, polycarbonates, polyethylene oxides, polyurethanes, polystyrenes, polyamides, polyimides, polyvinyl acetates, polyvinyl chlorides and polyvinylidene chlorides, and/or copolymers such as acrylonitrile/butadiene/styrene copolymers, optionally containing paper fibers, textile fibers and/or mixtures thereof, which can be metallized or pigmented if required. The carrier layer or covering layer of the plaster can also consist of a combination of metal foil and polymer layer. The thickness of the carrier layer is preferably 3 to 100 μm in each case.

The matrix layer of the plaster containing the active ingredient may contain matrix-forming polymers, skin penetration enhancers, solubilizers, crosslinking agents, stabilizers, emulsifiers, preservatives, thickeners and/or other conventional auxiliary substances.

The matrix-forming polymer used preferably comprises at least one film-forming polymer selected from the group consisting of hydroxypropyl cellulose, carboxymethyl cellulose, polyethylenes, chlorinated polyethylenes, polypropylenes, polyurethanes, polycarbonates, polyacrylic acid esters, polyacrylates, polymethacrylates, polyvinyl alcohols, polyvinyl chlorides, polyvinylidene chlorides, polyvinylpyrrolidones, polyethylene terephthalates, polytetrafluoroethylenes, ethylene/propylene copolymers, ethylene/ethyl acrylate copolymers, ethylene/vinyl acetate copolymers, ethylene/vinyl alcohol copolymers, ethylene/vinyloxyethanol copolymers, vinyl chloride/vinyl acetate copolymers, vinylpyrrolidone/ethylene/vinyl acetate copolymers, rubbers, rubber-like synthetic homopolymers, copolymers or block polymers, silicones, silicone derivatives, preferably siloxane/methacrylate copolymers, cellulose derivatives, preferably ethyl cellulose or cellulose ethers, and mixtures thereof. If the layer containing the active ingredient serves at the same time as the adhesive layer, it preferably contains at least one of the adhesives listed above in addition to at least one of the polymers listed.

Solubilizers which can be used include N-methyl-2-pyrrolidone, laurylpyrrolidone, triethanolamine, triacetin, diethylene glycol monoethyl ether, derivatives of fatty acids or fatty alcohols, low-molecular polyhydric alcohols, for example propylene glycol or glycerol, and/or surface-active compounds.

If the plaster containing the active ingredient is built up according to the reservoir system, the reservoir membrane can be comprised of inert polymers such as polyethylenes, polypropylenes, polyvinyl acetates, polyamides, ethylene/vinyl acetate copolymers and/or silicones. The reservoir membrane makes it possible to achieve a controlled release of the active ingredient from the reservoir.

The plaster matrix or reservoir containing the active ingredient can also contain solvents such as water, ethanol, 1-propanol, isopropanol, a low-molecular polyhydric alcohol, for example propylene glycol or glycerol, or an ester, for example isopropyl myristate, surface-active compounds or mixtures thereof.

Stabilizers which can be used for the matrix or reservoir containing active ingredient include antioxidants, e.g. vitamin E, butylhydroxytoluene, butylhydroxyanisole, ascorbic acid or ascorbyl palmitate, and/or chelating agents, e.g. disodium ethylenediaminetetraacetate or potassium or sodium citrate.

The matrix or reservoir containing the active ingredient can also contain conventional skin penetration enhancers.

The plaster can also contain, in one or more layers, at least one plasticizer or skin penetration enhancer selected from the group consisting of long-chain alcohols such as dodecanol, undecanol or octanol, esters of carboxylic acids with polyethoxylated alcohols, diesters of aliphatic dicarboxylic acids such as adipic acid, medium-chain triglycerides of caprylic acid and/or capric acid, coconut fat, polyhydric alcohols such as 1,2-propanediol, esters of polyhydric alcohols, such as glycerol, with laevulinic acid or caprylic acid, and etherified polyhydric alcohols.

The detachable protective layer(s) of the active ingredient release regulator according to the invention, and the detachable protective layer of the plaster, if present, can be comprised of polyethylene, polyester, polyethylene terephthalate, polypropylene, polysiloxane, polyvinyl chloride or polyurethane and optionally of treated paper fibers, e.g. cellophane, and optionally have at least one silicone, fluorosilicone or fluorocarbon coating. In the case of an active ingredient release regulator detachably connected to the plater, both surfaces of the protective layer are preferably treated in this way.

The active ingredient release regulator according to the invention or the plaster can be produced by known processes comprising steps such as lamination, co-extrusion, punching, delamination, unwinding, cutting, rewinding, assembly and/or dose portioning (see Verpackungs-Rundschau 4/2002, 83-84).

### BRIEF DESCRIPTION OF THE DRAWINGS

The invention will be described in further detail hereinafter with reference to examples of illustrative preferred embodiments shown in the accompanying drawing figures in which:

FIG. 1 is a schematic cross-sectional representation of a first release regulating system according to the invention which is initially separate from a plaster, and

FIG. 2 is a schematic cross-sectional representation of a second release regulator embodiment in accordance with the present invention which is already detachably bonded to a plaster.

### DETAILED DESCRIPTION

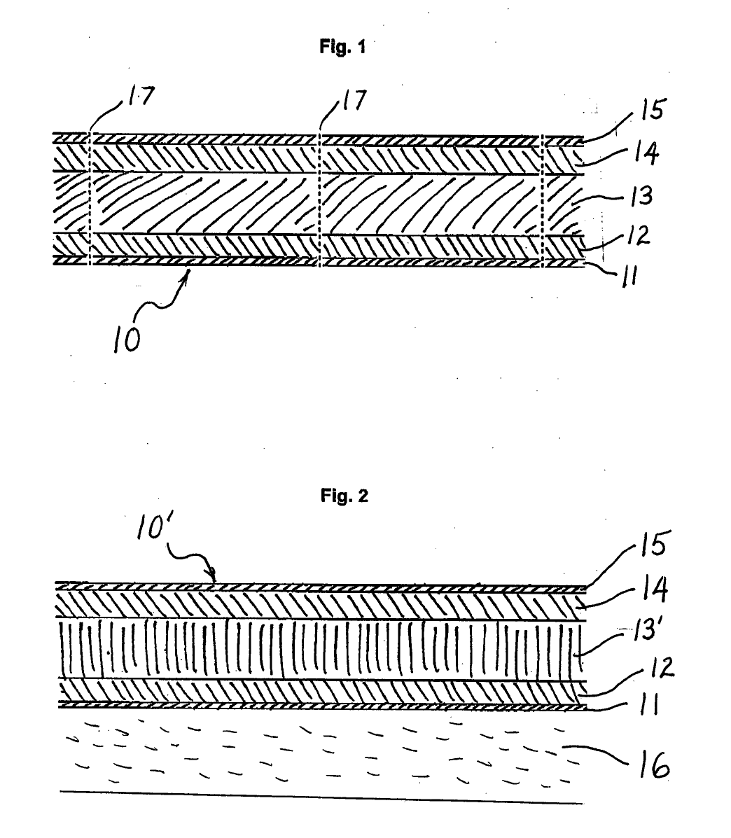

FIG. 1 illustrates a first representative release regulating system embodiment 10 according to the present invention which is initially separate from a plaster with which it is used. Regulating system 10 comprises a divisible, multi-layer active ingredient release regulator. The release regulator comprises a detachable protective layer 11, an adhesive layer 12, an active ingredient barrier layer 13, a second adhesive layer 14, and a second detachable protective layer 15. The divisibility of the regulator that is impermeable to the active ingredient is indicated by the vertical broken lines 17 which mark the points where the regulator can be readily divided. The barrier layer 13 may be one that is impermeable to the active ingredient, or it could alternatively be one which merely retards the release of the active ingredient from the plaster.

FIG. 2 schematically depicts a second representative embodiment of a release regulating system 10′ according to the present invention that is already detachably bonded to an active ingredient-containing plaster 16. This embodiment comprises a detachable protective layer 11, an adhesive layer 12, a barrier layer 13′ that optionally further retards the release of the active ingredients, a second adhesive layer 14, and a second detachable protective layer 15. The barrier layer 13′ may be one that retards the release of the active ingredient from a plaster, or it could alternatively be one which is impermeable to the active ingredient from the plaster 16. Although points of division are not illustrated in this drawing figure, it should be understood that this embodiment is also optionally divisible to vary the magnitude of its release retarding effects.

### EXAMPLES

### Example 1

a) Production of a Plaster Containing Buprenorphine

1139 g of a 48 wt. % polyacrylate solution of a self-crosslinking acrylate copolymer of 2-ethylhexyl acrylate, vinyl acetate and acrylic acid (solvent=ethyl acetate:heptane:isopropanol:toluene:acetylacetonate in proportions of 37:26:26:4:1), 100 g of laevulinic acid, 150 g of oleyl acetate, 100 g of polyvinylpyrrolidone, 150 g of ethanol, 200 g of ethyl acetate and 100 g of buprenorphine base were homogenized. The mixture was stirred for about two hours and checked visually that all the solids had dissolved. The evaporation loss was also checked by re-weighing and any lost solvent was replenished with ethyl acetate.

As covering layer, a transparent polyester sheet 420 mm wide was coated with the aforedescribed mixture so that the weight per unit area of the dried adhesive layer was 80 g/m². The solvents were removed by drying with warm air, which was passed over the moist strip. The heat treatment evaporated the solvents. Finally, the adhesive film containing active ingredient was covered with a polyester sheet 15 μm thick, which was made redetachable by a silicone treatment. An area for the intended amount of active ingredient was punched out with suitable cutting tools.

b) Production of an Active Ingredient Release Regulator with the Following Layer Structure:

a detachable protective layer
an adhesive layer
an active ingredient barrier layer
an adhesive layer
a detachable protective layer.

To produce the active ingredient release regulator according to the invention, a polyethylene terephthalate sheet 75 μm thick as the active ingredient barrier layer was clamped in an Ericsson film drawing machine (from Ericsson GmbH & Co. KG, Herma, Germany), coated with the mixture described in Example 1 a), with the exception of buprenorphine, and dried for 2 hours, to produce an adhesive layer 90 μm thick. This adhesive layer was bonded to a polyethylene terephthalate-based sheet treated with silicone on one side, detachably connected to the silicone-treated side. The process was repeated on the still free, second surface of the barrier layer so that both sides of the active ingredient barrier layer had an adhesive layer 90 μm thick and a protective layer detachably bonded thereto.

A suitable punching tool was used not only to punch out the active ingredient release regulator in a size to match that of the plaster produced according to Example 1 a), but also to create, by means of perforation lines, four squares of equal size on the active ingredient release regulator. By removing a quarter of the active ingredient release regulator along the provided perforation lines and bonding an adhesive layer of the remaining active ingredient release regulator, after removal of the detachable protective layer, to the region of the plaster produced in Example 1 a) that contained the active ingredient, it was possible to reduce the dosage and hence the release of the active ingredient by one-fourth.

### Example 2

a) Production of a Buprenorphine Plaster

The plaster was produced as described in Example 1 a).

b) Production of an Active Ingredient Release Regulator with the Following Layer Structure:

a detachable protective layer
an adhesive layer
a detachable protective layer.

To produce the active ingredient release regulator according to the invention, a polyethylene terephthalate sheet 36 μm thick treated with silicone on one side as detachable protective layer was clamped in an Ericsson film drawing machine (from Ericsson GmbH & Co. KG, Herma, Germany), coated with the mixture described in Example 1 a), with the exception of buprenorphine, and dried for 2 hours, to produce an adhesive layer 15 μm thick. This adhesive layer was detachably bonded to a polyethylene terephthalate-based sheet treated with silicone on one side.

Using a suitable punching tool, the active ingredient release regulator was punched out in a size corresponding to the plaster produced in Example 2 a). After removal of the detachable protective layer from the adhesive layer of the plaster produced in Example 2 a), and removal of a detachable protective layer from the adhesive layer of the active ingredient release regulator produced in Example 2 b), the exposed adhesive layers were bonded together. In this way the region of the plaster that contained the active ingredient was 100% covered with the regulator.

The efficacy of the active ingredient release regulator was evaluated by the tail flick test, i.e. the pharmacological pain evaluation test on rats. The test was performed with plasters according to 2 a) without combined use of an active ingredient release regulator, and with plasters according to 2 a) with combined use of an active ingredient release regulator according to 2 b). The corresponding test results are collated in the following table.

| | Buprenorphine | Buprenorphine plaster with |
|---|---|---|
| | plaster | active ingredient release |
| | without regulator | regulator |
| After 2 hours | no measurement | 33% MPE |
| After 4 hours | 95% MPE | 61% MPE |
| After 6 hours | 96% MPE | 81% MPE |
| After 8 hours | 100% MPE | 100% MPE |
| After 12 hours | 98% MPE | 100% MPE |

Data reported in percent of MPE (=maximum possible effect), a maximum analgesic action corresponding to 100% MPE.

The results clearly show that the release of the active ingredient is delayed in the first six hours following application.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. An active ingredient release system for systemic or topical release of an active ingredient to or through the skin of a human or animal organism, said release system comprising:
a) a plaster containing the active ingredient, and
b) an active ingredient release regulator selected from the group consisting of:
at least one divisible active ingredient release regulator that is impermeable to the active ingredient;
at least one optionally divisible active ingredient release regulator that retards the release of the active ingredient and is present separate from the plaster; and
an optionally divisible active ingredient release regulator that retards the release of active ingredient and that is detachably connected to the plaster.

2. An active ingredient release system according to claim 1, wherein the release regulator is a separately present, divisible active ingredient release regulator that is impermeable to the active ingredient and that comprises:
a detachable protective layer
an adhesive layer, and
an active ingredient barrier layer.

3. An active ingredient release system according to claim 2, wherein the release regulator further comprises a second adhesive layer and a second detachable protective layer

4. An active ingredient release system according to claim 2, wherein the release regulator is a divisible active ingredient release regulator impermeable to active ingredient and separate from the plaster and which comprises:
a detachable protective layer
an adhesive layer
an active ingredient barrier layer
a second adhesive layer for bonding to the skin, and
a second detachable protective layer.

5. Active ingredient release system according to claim 1, wherein the release regulator is an optionally divisible active ingredient release regulator that retards the release of active ingredient and that comprises:
a detachable protective layer, and
an adhesive layer.

6. An active ingredient release system according to claim 5, wherein said adhesive layer retards the release of the active ingredient.

7. An active ingredient release system according to claim 5, wherein said release regulator further comprises a layer that further retards the release of active ingredient; a second adhesive layer, and a second detachable protective layer.

8. An active ingredient release system according to claim 5, wherein the optionally divisible active ingredient release regulator that retards release of active ingredient is separate from the plaster and comprises the following layer structure:
a first detachable protective layer
a first adhesive layer
a layer that retards the release of the active ingredient
a second adhesive layer, and
a second detachable protective layer.

9. An active ingredient release system according to claim 5, wherein the optionally divisible active ingredient release regulator is separate from the plaster that retards the release of active ingredient and comprises the following layer structure:
a detachable protective layer
an adhesive layer that also retards the release of the active ingredient, and
a second detachable protective layer.

10. An active ingredient release system according to claim 5, wherein the optionally divisible active ingredient release regulator that retards the release of active ingredient is already detachably bonded to the plaster and comprises the following layer structure:
a first detachable protective layer,
a layer that retards the release of active ingredient,
an adhesive layer for bonding to the skin, and
a second detachable protective layer.

11. An active ingredient release system according to claim 10, wherein the release regulator further comprises an adhesive layer between the first detachable protective layer and the layer that retards the release of active ingredient.

12. An active ingredient release system according to claim 1, wherein the release regulator is an optionally divisible active ingredient release regulator that retards the release of active ingredient and is already detachably bonded to the plaster and comprises the following layer structure:
a detachable protective layer,
an adhesive layer that also retards the release of the active ingredient, and
a second detachable protective layer.

13. An active ingredient release system according to claim 1, wherein the active ingredient release regulator is divisible.

14. An active ingredient release system according to claim 13, wherein the divisible active ingredient release regulator is provided with separating means for facilitating division at predetermined locations.

15. An active ingredient release system according to claim 14, wherein said separating means is selected from the group consisting of lines of weakness, perforation lines, and cutting marks.

16. An active ingredient release system according to claim 1, wherein the active ingredient release regulator is impermeable to the active ingredient and is divisible so that from 10 to 90% of the surface of the region of the plaster that contains the active ingredient can be covered.

17. An active ingredient release system according to claim 1, comprising at least one active ingredient release regulator separate from the plaster.

18. An active ingredient release system according to claim 17, comprising at least two separate active ingredient release regulators of variable size.

19. An active ingredient release system according to claim 18, wherein the parts of a divisible active ingredient release regulator that can be separated from one another.

20. An active ingredient release system according to claim 19, wherein the divisible parts of the active ingredient release regulator are identifiable by different distinguishable colorings.

21. An active ingredient release system according to claim 18, wherein the active ingredient release regulators are packaged separately from one another and carry different distinguishing marks.

22. An active ingredient release system according to claim 21, wherein the separately packaged active ingredient release regulators are identifiable by different distinguishable colorings.

23. An active ingredient release system according to claim 1, wherein the active ingredient release regulator before application initially detachably covers the entire region of the plaster that contains the active ingredient.

24. An active ingredient release system according to claim 19, wherein from 10 to 100% of the region of the plaster that contains the active ingredient can be covered by the divisible active ingredient release regulator.

25. An active ingredient release system according to claim 21, wherein from 10 to 100% of the region of the plaster that contains the active ingredient can be covered by each of the separately packaged active ingredient release regulators.

26. An active ingredient release system according to claim 1, wherein said plaster comprises a reservoir or a matrix that contains the active ingredient.

27. An active ingredient release system according to claim 1, wherein the active ingredient release regulator is in the form of a kit.
